(19) United States
(12) Patent Application Publication
(10) Pub. No.: US 2024/0200094 A1
(43) Pub. Date: Jun. 20, 2024

(54) METHODS FOR PRODUCING SINGLE INSECT CELL CLONES

(71) Applicant: uniQure biopharma B.V., Amsterdam (NL)

(72) Inventors: Fiona Amanda Lucie MORENO, Amsterdam (NL); Anggakusuma, Amsterdam (NL); Hugo Federico CUETO ROJAS, Amsterdam (NL)

(73) Assignee: uniQure biopharma B.V., Amsterdam (NL)

(21) Appl. No.: 18/375,392

(22) Filed: Sep. 29, 2023

Related U.S. Application Data
(63) Continuation of application No. PCT/EP2022/058741, filed on Apr. 1, 2022.

(30) Foreign Application Priority Data
Apr. 2, 2021 (EP) 21166830.6

(51) Int. Cl. C12N 15/86; C12N 5/07

(52) U.S. Cl. CPC C12N 15/86; C12N 5/0601; C12N 2710/14122; C12N 2710/14143; C12N 2710/14144; C12N 2750/14122; C12N 2750/14143; C12N 2750/14152

(57) ABSTRACT

The present invention relates to a method for producing a single cell clone of an insect cell comprising integrated into the genome of the cell at least one expression cassette for inducible expression of parvoviral Rep 78 and 52 proteins. The insect cells can be used for the production of parvoviral vectors by transfection constructs comprising parvoviral Cap and ITR sequences. Single cell clones obtained in the methods of the invention can be expanded for into a cell bank for the production of clinical grade parvoviral gene therapy vectors.

### REFERENCE TO AN ELECTRONIC SEQUENCE LISTING

The contents of the electronic sequence listing (sequencelisting.xml; Size: 54.5 Kb; and Date of Creation: December 29, 2023) is herein incorporated by reference in its entirety.

### FIELD OF THE INVENTION

The present invention relates to the fields of medicine, molecular biology, gene therapy and insect cell culture. The invention relates to methods for producing single cell clones of insect cells that are used for the production of parvoviral gene therapy vectors.

### BACKGROUND OF THE INVENTION

In 2009, Aslanidi et al. (Proc Natl Acad Sci U S A. 2009;106(13):5059-64) created an Sf9 insect cell-based Rep-Cap packaging cell line that could produce adeno-associated virus (AAV) at 10⁵ genome copies (GC) per cell upon a single inoculation of baculovirus (Bac) harbouring the AAV inverted terminal repeats (ITR) and transgene of interest (Trans). This system, referred to as the OneBac platform, was considered to be suitable for the scale up of AAV production (Mietzsch, et al., 2014; Mietzsch, et al., 2017). Mietzsch et al. (2015) further optimized this platform to produce multiple AAV serotypes with low host-DNA false packaging. In a recent study, Wu et al. (2019) have shown that the OneBac platform can be more versatile and flexible by fusing the Cap gene together with the ITR-transgene-ITR (Cap-Trans) inside the baculovirus vector genome while maintaining the inducible Rep gene integrated within the packaging Rep Sf9 insect cells. All these experiments have shown the value of the OneBac platform and at the same time the necessity and possibility of improvement.

Smith et al., 2000 (WO 00/20561) described the generation of the Express Sf+cell line as being generated using several rounds of adaptation in serum free media, limited dilution and cell expansion, although monoclonality or establishment of an isogenic population was not achieved. Later work from Ma, H., et al., 2019 (Virology, 536: 125-133) in the cell line Sf9 reported that it was difficult if not nearly impossible for the authors to produce single cell clones because single cell clones tended to die and the limited dilution protocol was performed to the lowest cell count possible, which was about 30 cells per well, assuming an isogenic population. The same authors also demonstrate that the Sf9 cell line is a heterogenic population as shown by the presence of the genes of the Sf-associated rhabdovirus. However, after transfection, a mixed-population of genetically different cells has been obtained, also known as stable pool. Since the transgene can exist in different numbers of copies and can be integrated at different location in the genome, the cell pool contains cells with different levels of transgene expression and difference in growth performance.

Fernandes et al., 2012 (Biotechnol. Bioeng., 109: 2836-2844) showed that by using a mixture of filtered-media obtained from exponentially growing cells supplemented with 10% v/v fetal bovine serum, it was possible to establish isogenic populations of Sf9 cells. Therefore, there is still a need to generate a single insect cell (ExpresSf+) clone from a cells suspension in order to obtain a monoclonal homogeneous cell line, essentially, to overcome limitation associated with process robustness. Thus it is an object of the present invention to provide for means and methods that allow for high-yield, robust, and scalable production of heterologous parvoviral-related proteins and vectors.

### SUMMARY OF THE INVENTION

In one aspect, the invention relates to a method for producing a single cell clone of an insect cell comprising integrated into the genome of the cell at least one expression cassette for inducible expression of parvoviral Rep 78 and 52 proteins, the method comprising the steps of: a) preparing a series of sequential dilutions from a culture comprising a plurality of the insect cell, preferably in a conditioned medium; b) seeding an aliquot from a dilution obtained in a) into at least one culture container at a cell density of less than one insect cell per container; c) incubating the culture container under conditions that are conducive to growth of the insect cell and that do not induce expression of the parvoviral Rep 78 and 52 proteins; and, d) selecting at least one single cell clone from at least one culture container wherein the insect cells have grown.

In one embodiment of the method, in the step b) post-seeding the at least one culture container is inspected, preferably by visual inspection, to identify a culture container comprising no more than a single cell, and wherein step d) at least one single cell clone is selected from a culture container having been identified to comprise no more than a single cell.

In one embodiment of the method, the method further comprises the step of: e) inducing expression of the parvoviral Rep 78 and 52 proteins in a sample taken from at least one single cell clone obtained in d) and detecting induced expression of the parvoviral Rep 78 and 52 proteins, and wherein at least one single cell clone is selected wherein induced expression of the parvoviral Rep 78 and 52 proteins is detected.

In one embodiment of the method, the at least one expression cassette for inducible expression of the parvoviral Rep 78 and 52 proteins comprises at least one baculoviral promoter that is operably linked to at least one sequence coding for the parvoviral Rep 78 and 52 proteins, wherein the at least one baculoviral promoter is operably linked to a baculoviral homologous region (hr) enhancer element that is dependent on a baculoviral immediate-early protein (IE1) or its spice variant (IE0) as transcriptional transregulator, and wherein introduction of the baculoviral immediate-early protein (IE1) or its spice variant (IE0) into the cell induces transcription from the at least one baculoviral promoter.

In one embodiment of the method, the insect comprises integrated into the genome of the cell: i) a first baculoviral promoter operably linked to a nucleotide sequence encoding an mRNA, translation of which in the cell produces at least one of parvoviral Rep 78 and 68 proteins; ii) a second baculoviral promoter operably linked to a nucleotide sequence encoding an mRNA, translation of which in the cell produces at least one of parvoviral Rep 52 and 40 proteins; and iii) at least one hr enhancer element that is operably linked to the first and second promoters, wherein preferably the baculovirus is Autographa californica multicapsid nucleopolyhedrovirus. The construction of such insect cells, referred to as iRep cells, is described in detail in WO 2019/198510.

In one embodiment of the method, the hr enhancer element comprises at least one copy of the hr 28-mer sequence CTTTACGAGTAGAATTCTACGCGTAAAA and/or at least one copy of a sequence of which at least 20, 21, 22, 23, 24, 25, 26, or 27 nucleotides are identical to sequence CTTTACGAGTAGAATTCTACGCGTAAAA and which binds to a baculoviral IE1 protein, and wherein the hr enhancer element, when operably linked to an expression cassette comprising a reporter gene operably linked to the polH promoter, a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and, b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element, wherein preferably the hr enhancer element is selected from the group consisting of hr1, hr3, hr4b and hr5, of which hr4b and hr5 are preferred, of which hr4b is preferred.

In one embodiment of the method, the first and second promoters are distinct, wherein, preferably the first promoter is a delayed early baculoviral promoter and the second promoter is a late or very late baculovirus promoter, more preferably the first promoter is the 39k promoter and the second promoter is selected from the group consisting of the polH, p10, p6.9 and pSel120 promoters.

In one embodiment of the method, the at least one of parvoviral Rep 52 and 40 proteins and the at least one of parvoviral Rep 78 and 68 proteins have a common amino acid sequence that is at least 90% identical, while the nucleotide sequence encoding the common amino acid sequence in the mRNA for the at least one of parvoviral Rep 52 and 40 proteins has less than 95, 90, 85, 80, 75, 70, 65 or 60% sequence identity with the nucleotide sequence encoding the common the amino acid sequence in the mRNA for the at least one of parvoviral Rep 78 and 68 proteins, wherein preferably the codon usage in the nucleotide sequence encoding the common the amino acid sequence in the mRNA for the at least one of parvoviral Rep 52 and 40 proteins, is more adapted to the codon usage bias of the insect cell than codon usage in the nucleotide sequence encoding the common the amino acid sequence in the mRNA for the at least one of parvoviral Rep 78 and 68 proteins.

In one embodiment of the method, the nucleotide sequence encoding the mRNA for the at least one of parvoviral Rep 78 and 68 proteins comprises a modification that affects a reduced steady state level of the at least one of parvoviral Rep 78 and 68 proteins, preferably the at least one of parvoviral Rep78 and 68 proteins comprises an open reading frame that starts with a suboptimal translation initiation codon, wherein more preferably, the suboptimal translation initiation codon is selected from ACG, CTG, TTG, GTG and ATT, of which ACG is most preferred.

In one embodiment of the method, the first and second promoters are integrated in the cell's genome in opposite directions of transcription and wherein the at least one enhancer element is present in between the first and second promoters, wherein preferably two enhancer elements are present in between the first and second promoters.

In one embodiment of the method, in step e) the expression of the parvoviral Rep 78 and 52 proteins is induced by introducing into the cells of the single cell clone a first nucleotide sequence comprising an expression cassette for expression of the a baculoviral immediate-early protein (IE1) or its spice variant (IE0) transcriptional transregulator, wherein preferably, the first nucleotide sequence is comprised in a baculoviral vector.

In one embodiment of the method, in step e) simultaneous with the introduction of the first nucleotide sequence a second and a third nucleotide sequence is introduced into the cells of the single cell clone, wherein a) a second nucleotide sequence comprises parvoviral capsid protein coding sequences operably linked to a third promoter for expression in the insect cell; and b) a third nucleotide sequence comprising a transgene that is flanked by at least one parvoviral inverted terminal repeat sequence, wherein preferably, the second and third nucleotide sequences are comprised in at least one baculoviral vector, wherein more preferably the first, second and third nucleotide sequences are comprised together in a single baculoviral vector, and wherein optionally, in step e) and the production parvoviral particles is detected, wherein a single cell clone wherein production of parvoviral particles is detected is selected.

In one embodiment of the method, at least one of: a) the insect cell is an insect cell selected from the group consisting of ExpresSf+™, Sf9, Sf21, TN-368, BTITN-5BI-4 and High-Five™; and, b) the conditioned medium comprises spent insect cell medium, wherein preferably the spent medium is spent by an insect cell that is an untransformed predecessor of the insect cell, and wherein more preferably the conditioned medium is supplemented with at least one of fetal bovine serum and L-glutamine.

In a further aspect, the invention relates to a method for producing a cell bank of a single cell clone of an insect cell comprising integrated into the genome of the cell at least one expression cassette for inducible expression of parvoviral Rep 78 and 52 proteins, the method comprising the steps of: a) producing and/or selecting a single cell clone in a method according to the invention; b) expanding the single cell clone obtained in a); and, c) distributing the expanded single cell clone obtained in b) over a plurality of vials, and optionally, storage of the vials.

In yet a further aspect, the invention relates to a single cell clone produced and/or selected in a method according to the invention, or a cell bank consisting of a plurality of vials of the single cell clone obtained in a method according to the invention.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6×sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### DETAILED DESCRIPTION OF THE INVENTION

The inventors identified that typical conditions discussed in the prior art, such as using spent media, FBS supplementation and insulin supplementation were not sufficient to produce single cell clones in insect cell lines, such as Express Sf+, which is a derived cell line from Sf9. Therefore, they have set out to develop methods for producing a single cell clone of an insect cell since normally, the cell pool contains cells with different levels of transgene expression and difference in growth performance. A monoclonal homogeneous cell line therefore has the same levels of transgene expression and growth performance and can be used reliably in, for example, AAV vector production and in upscaling processes.

In one aspect, the invention relates to a method for producing a single cell clone of an insect cell. Preferably the insect cell is an insect cell to be used for the production of a biomolecule, such as a polypeptide, nucleic acid or a virus. Usually the insect cell will therefore be a genetically modified insect cell, or a non-naturally occurring insect cell.

The term “clone” as used herein refers to a cell population derived from a single cell, i.e. a single cell clone. A monoclonal population of cells derived from a single cell will thus be a homogeneous isogenic population. Therefore, a pool of clones comprise cells with the same, or virtually the same, DNA.

In one embodiment, the method of the invention for producing a single cell clone of an insect cell that is an insect cell for producing a parvoviral vector, preferably a parvoviral vector for gene therapy such as an AAV gene therapy vector.

The insect cell can be any cell that is suitable for the production of heterologous proteins. Preferably the insect cell allows for replication of baculoviral vectors and can be maintained in culture. More preferably the insect cell also allows for replication of recombinant parvoviral vectors, including rAAV vectors. For example, the cell line used can be from Spodoptera frugiperda, Drosophila cell lines, or mosquito cell lines, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). A preferred insect cell according to the invention is an insect cell for production of recombinant parvoviral vectors. In one embodiment of the method of the invention, at least one of: a) the insect cell is an insect cell selected from the group consisting of ExpresSf+™, Sf9, Sf21, TN-368, BTITN-5BI-4 and High-Five™; and, b) the conditioned medium comprises spent insect cell medium, wherein preferably the spent medium is spent by an insect cell that is an untransformed predecessor of the insect cell, and wherein more preferably the conditioned medium is supplemented with at least one of fetal bovine serum and L-glutamine. In a preferred embodiment of the method of the invention, at least one of: a) the insect cell is a ExpresSf+™ cell or a cell derived therefrom; and, b) the conditioned medium comprises spent insect cell medium, wherein preferably the spent medium is spent by an insect cell that is an untransformed predecessor of the insect cell, and wherein more preferably the conditioned medium is supplemented with at least one of fetal bovine serum and L-glutamine.

In one embodiment, the method of the invention for producing a single cell clone of an insect cell comprises one or more of limiting dilution, automated clone picking, use of cloning rings or fluorescence activated cell sorting. In one embodiment, the method of the invention for producing a single cell clone of an insect cell comprises limiting dilution. The term “limiting dilution” and “dilution cloning” as used herein refers to a procedure to obtain a monoclonal cell population starting from a polyclonal mass of cells. This is achieved by setting up a series of increasing dilutions of the parent (polyclonal) cell culture. A suspension of the parent cells is made. Appropriate dilutions are then made, depending on cell number in the starting population, as well as the viability and characteristics of the cells being cloned. In one embodiment the dilutions are made only after a cell confluency of 50%, 60%, 70%, 80% or 90%, more preferably dilutions are made only after a cell confluency of 80%.

In one embodiment, the conditioned medium comprises spent insect cell medium, insect cell media, fetal bovine serum and L-glutamine. In a further embodiment, the condition medium comprises between 50 to 90% (v/v) spent insect cell medium, between 5 to 15% (v/v) fetal bovine serum, and between 2 to 20 mM L-glutamine. In a further embodiment, the condition medium comprises between 70 to 85% (v/v) spent insect cell medium, between 7 to 12% (v/v) fetal bovine serum, between 5 to 15 mM L-glutamine. In a further embodiment, the condition medium comprises 85% (v/v) spent insect cell medium, 10% (v/v) fetal bovine serum, 10 mM L-glutamine. The volume of the conditioned medium can be complemented to 100% (v/v) with fresh insect cell medium and/or the volumes of the glutamine stock solution (e.g. 200 mM L-glutamine).

One of ordinary skill in the art would be equipped to select the cell medium suitable for the cell being cultured. For example, Sf900 II SFM (Thermo Fischer Scientific, catalogue number 10902096) is a suitable medium for SF+cells, SF9 and derived cell lines. The term “spent cell medium” and “spent insect cell medium” as used herein would be known to one in the art to be a cell medium that is formed by cell growth and remains after culturing of cells. Preferably, the spent cell medium for use in the method of the invention is obtained by growing an insect cell of the same lineage or strain as the insect cell from which a single cell clone is produced. For example, an untransformed parental insect cell can be used to prepare spent cell medium that is used to obtain a single cell clone of a transformed insect cell.

In one embodiment, the spent cell medium is produced by seeding insect cells into fresh insect cell medium at a density of 2.5−6×10⁶ viable insect cells/mL, incubating for about 48 hrs under standard conditions for insect cell growth and harvesting the spent cell medium by separating the medium from the cells (e.g. by centrifugation), and optionally filter sterilization of the medium. One of ordinary skill in the art would be equipped to select similar or equivalent conditions for growing the cells to produce a spent cell medium.

One of ordinary skill in the art knows how to stably introduce a nucleotide sequence into the insect genome and how to identify a cell having such a nucleotide sequence in the genome. The incorporation into the genome may be aided by, for example, the use of a vector comprising nucleotide sequences highly homologous to regions of the insect genome. The use of specific sequences, such as transposons, is another way to introduce a nucleotide sequence into a genome. The incorporation into the genome may be through one or more than one steps. Reference to the term “integrated” will be known to one in the art to also mean “stably integrated”.

Growing conditions for insect cells in culture, and production of heterologous products in insect cells in culture are well-known in the art and described e.g. in the above cited references on molecular engineering of insect cells (see also WO2007/046703). In one embodiment, the cell growing conditions comprise a CO₂ level fixed above 0.1%, such as 0.5%, 1%, 1.5%, 2%, 2.5%, 3% or greater, preferably 1%. In one embodiment, the cell growing conditions comprise a temperature of between 25° C. and 35° C., preferably between 27.5° C. and 28.5° C., such as 28° C.

An “insect cell-compatible vector” or “vector” is understood to be a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the nucleic acid construct is a baculovirus-expression vector. Baculovirus-expression vectors and methods for their use are described for example in: Summers and Smith, 1986, “A Manual of Methods for Baculovirus Vectors and Insect Culture Procedures”, Texas Agricultural Experimental Station Bull. No. 7555, College Station, Tex.; Luckow, 1991, In Prokop et al., “Cloning and Expression of Heterologous Genes in Insect Cells with Baculovirus Vectors' Recombinant DNA Technology and Applications”, 97-152; King and Possee, 1992, “The baculovirus expression system”, Chapman and Hall, United Kingdom; O′Reilly, Miller, and Luckow, 1992, “Baculovirus Expression Vectors: A Laboratory Manual”, New York; Freeman and Richardson, 1995, “Baculovirus Expression Protocols”, Methods in Molecular Biology, volume 39; U.S. Pat. No. 4,745,051; US2003148506; and WO 03/074714.

The number of nucleic acid constructs employed in the insect cell for the production of the recombinant parvoviral (rAAV) vector is not limiting in the invention. For example, one, two, three or more separate constructs can be employed to produce rAAV in insect cells in accordance with the methods of the present invention. If two constructs are employed, one construct may comprise the nucleotide sequence comprising the transgene that is flanked by at least one parvoviral ITR sequence and the other construct may then comprise expression cassettes for respectively the Rep and Cap proteins. If three constructs are employed, one construct may comprise the nucleotide sequence comprising the transgene that is flanked by at least one parvoviral ITR sequence, another construct may comprise an expression cassette for the Cap proteins and still another construct may comprise one or more expression cassettes for the Rep protein, e.g. two expression cassettes, one for each of the Rep 78 and 52 proteins, optionally either codon optimized, AT-optimized or GC-optimized, in order to minimize or prevent recombination, as described hereinafter. It is hereby understood that at least some of the nucleic acid constructs, preferably those comprising the one or more expression cassettes for the Rep proteins, can be stably integrated into the genome of the insect cell.

In one embodiment, the method of the invention for producing a single cell clone of an insect cell that is an insect cell for producing a parvoviral vector, wherein the insect cell has been modified to comprise at least one expression cassette for expression of parvoviral Rep 78 and 52 proteins. Preferably, the insect cell has been modified to comprise at least one expression cassette for inducible expression of at least one of parvoviral Rep 78 and 52 proteins.

In one embodiment, the insect cell for producing a parvoviral vector has been modified to comprise a single (type of) expression cassette for expression of both parvoviral Rep 78 and 52 proteins from a single Rep 78 coding sequence. This can e.g. be effected by using a suboptimal initiation codon for the Rep 78 coding sequence, that effect partial exon skipping, allowing the ribosome to initiate translation at the Rep 52 initiation codon. Alternatively this can be effected by e.g. inserting an intron comprising a second promoter in the Rep 78 coding sequence upstream of the Rep 52 initiation codon.

In another embodiment, the insect cell for producing a parvoviral vector has been modified to comprise separate expression cassette for expression of each of the parvoviral Rep 78 and 52 proteins as further detailed herein below.

In one embodiment, the insect cell for producing a parvoviral vector has been modified to comprise at least one of the above-mentioned expression cassettes for expression of the parvoviral Rep 78 and/or 52 proteins, integrated into the genome of the insect cell.

In one embodiment, the method of the invention for producing a single cell clone of an insect cell at least comprises the steps of: a) preparing a series of sequential dilutions from a culture comprising a plurality of the insect cell, preferably in a conditioned medium; b) seeding an aliquot from a dilution obtained in a) into at least one culture container at a cell density of less than one insect cell per container; c) incubating the culture container under conditions that are conducive to growth of the insect cell; and, d) selecting at least one single cell clone from at least one culture container wherein the insect cells have grown.

In one embodiment, less than 10 sequential dilutions are prepared, such as 9, 8, 7, 6, 5, 4, 3, 2 serial dilutions, preferably 4 sequential dilutions are prepared. In one embodiment the desired cell density is between 2.5 and 25000 cells/mL, such as 2.5, 25, 250, 2500 and 25000 cells/mL for each sequential dilution.

In one embodiment the incubation in step c) is on plates prepared for culture, such as a 4 or 6 or 12, 24, 48 or 96 well plate or a T25, T75, T175 or T225 flask or a 25 mm, 60 mm 100 mm or 150 mm dish. In one embodiment the culture conditions of step c) are 28° C. and 135 rpm. In one embodiment step c) is maintained for 2, 3, 4 or 5 days, preferably between 2 and 3 days. In one embodiment steps b) and c) are repeated for 1 or more cycles, such as 2, 3, 4, 5, 6, 7, 8, 9 or 10 or more cycles. Consequently, in one embodiment, steps b) and c) may be carried out over a total of 5 to 10 days, preferably carried out in a total of 10 days.

In one embodiment, wherein the insect cell has been modified to comprise at least one expression cassette for inducible expression of at least one of parvoviral Rep 78 and 52 proteins, in step c) of the method, the culture container is preferably incubated under conditions that are conducive to growth of the insect cell and that do not induce expression of the parvoviral Rep 78 and 52 proteins.

In one embodiment of the method of the invention, in step b) post-seeding the at least one culture container is inspected to identify a culture container comprising no more than a single cell, and wherein step d) at least one single cell clone is selected from a culture container having been identified to comprise no more than a single cell. The at least one culture containers are preferably inspected visually, e.g. using a microscope. In one embodiment at least one culture container is inspected for the presence of only a single cell using whole container imaging. Particularly, when the culture containers are individual wells in a microtiter plates, automated high contrast whole well imaging such as Cell Metric™ (from Solentim; www.solentim.com) is preferably applied for identification of culture containers/well in which only a single cell is present.

In one embodiment of the method of the invention, the method further comprises the step of: e) inducing expression of the parvoviral Rep 78 and 52 proteins in a sample taken from at least one single cell clone obtained in d) and detecting induced expression of the parvoviral Rep 78 and 52 proteins, and wherein at least one single cell clone is selected wherein induced expression of the parvoviral Rep 78 and 52 proteins is detected. In one embodiment of the method of the invention, the at least one expression cassette for inducible expression of the parvoviral Rep 78 and 52 proteins comprises at least one baculoviral promoter that is operably linked to at least one sequence coding for the parvoviral Rep 78 and 52 proteins, wherein the at least one baculoviral promoter is operably linked to a baculoviral homologous region (hr) enhancer element that is dependent on a baculoviral immediate-early protein (IE1) or its spice variant (IE0) as transcriptional transregulator, and wherein introduction of the baculoviral immediate-early protein (IE1) or its spice variant (IE0) into the cell induces transcription from the at least one baculoviral promoter.

### Promoters

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells. A “cryptic promoter” is an epigenetically silenced promoter which may be activated.

In a preferred embodiment, the ratio of expression of the Rep78 versus the Rep52 protein is regulated by one or more of the following: (a) the second promoter is stronger than the first promoter, as e.g. determined by reporter gene expression (e.g. luciferase or SEAP), or northern or western blot; (b) the presence of nucleotide spacer or more and/or stronger enhancer elements at upstream of the second expression cassette as compared to the first expression cassette; (c) the nucleotide sequence coding for the parvoviral Rep52 protein has a higher codon adaptation index as compared to the nucleotide sequence coding for the Rep78 protein; (d) temperature optimization of the parvoviral Rep protein; and (e) variant Rep proteins with one or more alterations in the amino acid sequence as compared to a corresponding wild-type Rep protein and wherein the one or more amino acid alteration result in increases in the activity of the Rep function as assessed by detecting increased AAV production in insect cells. Methods for generation, selection and/or screening of variant Rep proteins with increased activity of Rep function as assessed by detecting increased AAV production in insect cells may be obtained by adaptation to insect cells of the methods described in US20030134351 for obtaining variant Rep proteins with increased function with respect to AAV production in mammalian cells. Variant Rep proteins with one or more alterations in the amino acid sequence as compared to a corresponding wild-type Rep protein are herein understood to include Rep proteins with one or more amino acid substitutions, insertions and/or deletions in the variant amino acid sequence as compared to the amino acid sequence of a corresponding wild type Rep protein.

The second promoter being stronger than the first promoter means that more mRNA molecules coding for a Rep52 protein are expressed than mRNA molecules coding for a Rep78 protein. An equally strong promoter may be used, since expression of Rep52 protein will then be increased as compared to expression of Rep78 protein. The strength of the promoter may be determined by the expression that is obtained under conditions that are used in the method of the invention.

In one embodiment, the first and second promoters are baculoviral promoters. In one embodiment, the first and second promoters are distinct. In one embodiment, the first promoter is a delayed early baculoviral promoter, such as the 39k promoter. In one embodiment, the second promoter is a late or very late baculovirus promoter, such as the polH, p10, p6.9 and pSel120 promoters. Consequently, in one embodiment, the first promoter is a delayed early baculoviral promoter and the second promoter is a late or very late baculovirus promoter. Therefore, in one embodiment, the first promoter is the 39k promoter and the second promoter is selected from the group consisting of the p10, p6.9 and pSel120 promoters.

In one embodiment, the first and second promoters are integrated in the cell's genome in opposite directions of transcription.

As described below, for production of a complete parvoviral gene therapy vector virion, the cell preferably further comprises an expression cassette comprising a nucleotide sequence comprising parvoviral capsid protein coding sequences operably linked to a third promoter for expression in the insect cell. In one embodiment, the first, second and third promoters are baculoviral promoters. In one embodiment, the first, second and third promoters are distinct. In one embodiment, the first promoter is a delayed early baculoviral promoter, such as the 39k promoter. In one embodiment, the second promoter is a late or very late baculovirus promoter, such as the polH, p10, p6.9 and pSel120 promoters. Consequently, in one embodiment, the first promoter is a delayed early baculoviral promoter and the second promoter is a late or very late baculovirus promoter. Therefore, in one embodiment, the first promoter is the 39k promoter and the second promoter is selected from the group consisting of the polH, p10, p6.9 and pSel120 promoters. In one embodiment, the first promoter is active before the third promoter.

In one embodiment of the method of the invention, the insect comprises integrated into the genome of the cell: i) a first baculoviral promoter operably linked to a nucleotide sequence encoding an mRNA, translation of which in the cell produces at least one of parvoviral Rep 78 and 68 proteins; ii) a second baculoviral promoter operably linked to a nucleotide sequence encoding an mRNA, translation of which in the cell produces at least one of parvoviral Rep 52 and 40 proteins; and iii) at least one hr enhancer element that is operably linked to the first and second promoters, wherein preferably the baculovirus is Autographa californica multicapsid nucleopolyhedrovirus (AcMNPV).

### Enhancer

An “enhancer element” or “enhancer” is meant to define a sequence which enhances the activity of a promoter (i.e. increases the rate of transcription of a sequence downstream of the promoter) which, as opposed to a promoter, does not possess promoter activity, and which can usually function irrespective of its location and direction with respect to the promoter (i.e. upstream, or downstream of the promoter). Enhancer elements are well-known in the art. Non-limiting examples of enhancer elements (or parts thereof) which could be used in the present invention include baculovirus enhancers and enhancer elements found in insect cells. It is preferred that the enhancer element increases in a cell the mRNA expression of a gene, to which the promoter it is operably linked, by at least 25%, more preferably at least 50%, even more preferably at least 100%, and most preferably at least 200% as compared to the mRNA expression of the gene in the absence of the enhancer element. mRNA expression may be determined for example by quantitative RT-PCR.

Herein it is preferred to use an enhancer element to enhance the expression of parvoviral Rep proteins. In a preferred embodiment at least one enhancer element that is operably linked to the (first and/or second) promoter as defined herein that is operably linked to a nucleotide sequence encoding an mRNA, translation of which in the cell produces a parvoviral Rep protein, is an enhancer element that is dependent on a transcriptional transregulator. A transcriptional transregulator-dependent enhancer element is herein understood as an enhancer element that activates transcription of a promoter operably linked thereto when bound by a transcriptional transregulator protein provided in trans.

Thus, in a further preferred embodiment, the transcriptional transregulator-dependent enhancer element comprises at least one baculovirus enhancer element and/or at least one ecdysone responsive element. Preferably the transcriptional transregulator is a baculoviral immediate-early protein (IE1) or its spice variant (IE0) and the transcriptional transregulator-dependent enhancer element is a baculoviral homologous region (hr) enhancer element, wherein preferably the baculovirus is Autographa californica multicapsid nucleopolyhedronvirus. IE1 is a highly conserved, 67-kDa DNA binding protein that transactivates baculovirus early gene promoters and supports late gene expression in plasmid transfection assays (see e.g. Olson et al., 2002, J Virol., 76:9505-9515). AcMNPV IE1 possesses separable domains that contribute to promoter transactivation and DNA binding. The N-terminal half of this 582-residue phosphoprotein contains transcriptional stimulatory domains from residue 8 to 118 and 168 to 222. IE1 binds to the ˜28-bp imperfect palindrome (28-mer) that constitutes repetitive sequences within multiple homologous regions (hrs) found dispersed throughout the AcMNPV genome. The hr 28-mer is the minimal sequence motif required for IE1-mediated enhancer and origin-specific replication functions.

In one embodiment, the hr enhancer element is an hr enhancer element other than hr2-0.9 US 2012/100606 A1). In a further embodiment, the hr enhancer element is selected from the group consisting of hr1, hr3, hr4b and hr5, of which hr4b and hr5 are preferred, of which hr4b is most preferred. In an alternative embodiment, the hr enhancer element is a variant hr enhancer element, such as e.g. a non-naturally occurring designed element. The variant hr enhancer element preferably comprises least the at one copy of hr 28-mer sequence CTTTACGAGTAGAATTCTACGCGTAAAA (SEQ ID NO. 1) and/or at least one copy of a sequence of which at least 18, 20, 21, 22, 23, 24, 25, 26, or 27 nucleotides are identical to sequence CTTTACGAGTAGAATTCTACGCGTAAAA (SEQ ID NO. 1) and which preferably binds to the baculoviral IE1 protein, more preferably to the AcMNPV IE1 protein.

The variant hr enhancer element is further preferably functionally defined in that when the variant element is operably linked to an expression cassette comprising a reporter gene operably linked to the polH promoter, a) under non-inducing conditions, the cassette with the variant element produces less reporter transcript than an otherwise identical expression cassette which comprises the hr2-0.9 element instead of the variant element, or the cassette with the variant element produces less than a factor 1.1, 1.2, 1.5, 2, 5 or 10 of the amount reporter transcript produced by an otherwise identical expression cassette which comprises the hr4b element instead of the variant element; and b) under inducing conditions, the cassette with the variant element produces at least 50, 60, 70, 80, 90 or 100% of the amount of reporter transcript produced by an otherwise identical expression cassette which comprises the hr4b or the hr2-0.9 element instead of the variant element, wherein preferably the hr enhancer element is selected from the group consisting of hr1, hr3, hr4b and hr5, of which hr4b and hr5 are preferred, of which hr4b is preferred.

Non-inducing conditions are understood as condition in which there is no IE1 protein present in the cell wherein the cassettes are tested and inducing conditions are understood to be conditions wherein sufficient IE1 protein is present to obtain maximal reporter expression with the reference cassettes comprising the hr4b or the hr2-0.9 element. Binding of the variant hr enhancer element to the baculoviral IE1 protein can be assayed by using a mobility shift assay as e.g. described by Rodems and Friesen (J Virol. 1995; 69(9):5368-75).

In one embodiment, the at least one enhancer element is present in between the first and second promoters. Consequently, in one embodiment, the first and second promoters are integrated in the cell's genome in opposite directions of transcription and the at least one enhancer element is present in between the first and second promoters. In a further embodiment, two enhancer elements are present in between the first and second promoters. When using Bac polH Cap Trans for induction, a relatively weaker transactivation profile is observed due to i) the cis:trans promoter competition between the two polH promoters used (for Cap in the Bac polH Cap Trans and for Rep in the expression plasmid) and ii) the adoption of non-leaky but relatively weaker hr such as hr4b. In one embodiment, there is the use of such a non-leaky expression platform, for example, the use of a relatively weaker hr such as hr4b. In a further embodiment, there is compatibility with the use of Bac polH Cap Trans. In still a further embodiment, the hr4b enhancer is combined with the p10 promoter. Such a combination may be made to regulate single-cassette AAV2 Rep with a strong wild-type ATG start codon.

In one embodiment of the method of the invention, the first and second promoters are distinct, wherein, preferably the first promoter is a delayed early baculoviral promoter and the second promoter is a late or very late baculovirus promoter, more preferably the first promoter is the 39k promoter and the second promoter is selected from the group consisting of the polH, p10, p6.9 and pSel120 promoters.

### Replicase (Rep) Proteins

Parvoviral, especially AAV, replicases are non-structural proteins encoded by the rep gene. In wild type parvoviruses the rep gene produces two overlapping messenger ribonucleic acids (mRNA) with different length, due to an internal P19 promoter. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serve as a viral replication initiator proteins and act as replicase for the viral genome (Chejanovsky and Carter, J Virol., 1990, 64:1764-1770; Hong et al., Proc Natl Acad Sci USA, 1992, 89:4673-4677; Ni., et al., J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (Smith and Kotin, J. Virol., 1998, 4874-4881; King, et al., EMBO J., 2001, 20:3282-3291). To produce AAV from the baculoviral vectors in an insect cell platform, the presence of both Rep68 and Rep40 is not prerequisite (Urabe, et al., 2002).

A nucleotide sequence encoding a parvoviral Rep protein, is herein understood as a nucleotide sequence encoding at least one of the two non-structural Rep proteins, Rep 78 and Rep52, that together are required and sufficient for parvoviral vector production in insect cells. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 8 and 9) or primates (e.g., serotypes 1 and 4). An example of a nucleotide sequence encoding parvovirus Rep proteins is given in SEQ ID NO. 9 (see SEQ ID NO. 5 of WO 2009/104964, included herein by reference), which depicts a part of the AAV serotype-2 sequence genome encoding the Rep proteins. The Rep78 coding sequence comprises nucleotides 11-1876 and the Rep52 coding sequence comprises nucleotides 683-1876, also depicted separately in SEQ ID NOS. 9 and 11 (see SEQ ID NOS. 5 and 7 of WO 2009/104964, included herein by reference). It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2.

According to the invention, the cell preferably comprises integrated into its genome a first nucleic acid construct that comprises at least a first and a second expression cassette for expression of the parvoviral Rep proteins.

The first expression cassette comprises a first promoter operably linked to a nucleotide sequence encoding an mRNA, translation of which in the cell produces at least one of parvoviral Rep 78 and 68 proteins. A nucleotide sequence encoding an mRNA, translation of which in an insect cell produces at least one of parvoviral Rep 78 and 68 proteins can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 12 (see SEQ ID NO. 8 of WO 2009/104964, included herein by reference); b) that has at least 50, 60, 70, 80, 81, 82, 85, 90, 95, 97, 98, or 99% sequence identity with the nucleotide sequence of positions 11-1876 of SEQ ID NO. 11 (see SEQ ID NO. 7 of WO 2009/104964, included herein by reference); c) the complementary strand of which hybridises to a nucleic acid molecule sequence of (a) or (b); and d) nucleotide sequences the sequence of which differs from the sequence of a nucleic acid molecule of (c) due to the degeneracy of the genetic code. Preferably, the nucleotide sequence encodes an mRNA, translation of which in an insect cell produces only at least one of parvoviral Rep 78 and 68 proteins. It is understood that translation the mRNA in an insect cell will usually only produce at least a parvoviral Rep 78 protein and need not produce a parvoviral Rep 68 protein. It is further understood that while the nucleotide sequence encodes an mRNA, translation of which in an insect cell produces only at least one of parvoviral Rep 78 and 68 proteins (and not the parvoviral Rep 52 and 40 proteins), this does not exclude that the nucleotide sequence comprises an internal endogenous parvoviral P19 promoter that is active in insect cells and which produces an additional mRNA, translation of which in an insect cell produces at least one of parvoviral Rep 52 and 40 proteins. In a preferred embodiment, the nucleotide sequence that encodes the mRNA, translation of which in an insect cell produces only at least one of parvoviral Rep 78 and 68 proteins, indeed comprises a parvoviral P19 promoter, which preferably is intact or at least active in an insect cell.

The second expression cassette comprises a second promoter operably linked to a nucleotide sequence encoding an mRNA, translation of which in the cell produces at least one of parvoviral Rep 52 and 40 proteins. A nucleotide sequence encoding an mRNA, translation of which in an insect cell produces at least one of parvoviral Rep 52 and 40 proteins can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 10 (see SEQ ID NO. 6 of WO 2009/104964, included herein by reference); b) that has at least 50, 60, 70, 80, 81, 82, 85, 90, 95, 97, 98, or 99% sequence identity with the nucleotide sequence of any one of SEQ ID NOS. 5-9 (see SEQ ID NOS. 1-5 of WO 2009/104964, included herein by reference) and SEQ ID NO. 2, of which SEQ ID NO. 2 is preferred; c) the complementary strand of which hybridises to a nucleic acid molecule sequence of (a) or (b); and, d) nucleotide sequences the sequence of which differs from the sequence of a nucleic acid molecule of (c) due to the degeneracy of the genetic code. Preferably, the nucleotide sequence encodes an mRNA, translation of which in an insect cell produces only at least one of parvoviral Rep 52 and 40 proteins. Thereby it is understood that the nucleotide sequence encoding the parvoviral Rep 52 and/or 40 proteins is not part of a larger coding sequence that also encodes the parvoviral Rep 78 and/or 68 proteins. Preferably the nucleotide sequence encoding an mRNA, translation of which in the cell produces only at least one of parvoviral Rep 52 and 40 proteins comprises an open reading frame that consists of the amino acid sequence from the translation initiation codon to the most C-terminal amino acid of the at least one of parvoviral Rep 52 and 40 proteins, more preferably, the open reading frame is the only open reading frame comprised in the nucleotide sequence encoding an mRNA. It is further understood that translation the mRNA in an insect cell will usually only produce at least a parvoviral Rep 52 protein and need not produce a parvoviral Rep 40 protein.

Preferably, the nucleotide sequence encodes parvovirus Rep proteins that are functionally active in the sense that they have the required activities of viral replication initiator protein, replicase of the viral genome, DNA helicase and packaging of viral DNA into empty capsids as described above, sufficient for parvoviral vector production in insect cells.

In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In a preferred embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90, 91, 92, 93, 94, 95, 96, 97, 98, 99 or 100% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90, 89, 88, 87, 86, 85, 84, 83, 82, 81, 80, 79, 78, 77, 76, 75, 74, 73, 72, 71, 70, 69, 68, 67, 66, 60% identical.

In a further embodiment, the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40. Preferably, however, the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins.

The adaptiveness of a nucleotide sequence encoding the common amino acid sequence to the codon usage of the host cell can be expressed as codon adaptation index (CAI). Preferably the codon usage is adapted to the insect cell wherein Rep proteins with the common amino acid sequence are expressed. Usually this will be a cell of the genus Spodoptera, more preferably a Spodoptera frugiperda cell. The codon usage is thus preferably adapted to Spodoptera frugiperda or to an Autographa californica nucleopolyhedrovirus (AcMNPV) infected cell. A codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (Sharp and Li, 1987, Nucleic Acids Research 15: 1281-1295; also see: Kim et al., Gene. 1997, 25 199:293-301; zur Megede et al., Journal of Virology, 2000, 74: 2628-2635).

Preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.1, 0.2, 0.3, 0.4, 0.5, 0.6, 0.7 or 0.8, whereby more preferably, the CAI of the nucleotide sequence coding for the common amino acid sequence in the at least one of parvoviral Rep 52 and 40 proteins is at least 0.5, 0.6, 0.7, 0.8, 0.9 or 1.0.

Therefore, in an alternative embodiment of the method of the invention, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90% identical, and the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins, wherein preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.2. In a preferred embodiment the nucleotide sequence encoding the common amino acid sequence of the at least one of parvoviral Rep 52 and 40 proteins with an improved codon usage bias for the insect cell has a nucleotide sequence of which at least 80, 85, 90, 95, 96, 97, 98, 99 or 100% of the codons are identical to the codons in SEQ ID NO. 2.

Temperature optimization of the parvoviral Rep protein refers to use the optimal condition with respect to both the temperature at which the insect cell will grow and Rep is functioning. A Rep protein may for example be optimally active at 37° C., whereas an insect cell may grow optimally at 28° C. A temperature at which both the Rep protein is active and the insect cell grows may be 30° C. In a preferred embodiment, the optimized temperature is more than 27, 28, 29, 30, 31, 32, 33, 34 or 35° C. and/or less than 37, 36, 35, 34, 33, 32, 31, 30 or 29° C.

In one embodiment, the first and second expression cassettes in the cell are optimised to obtain a desired molar ratio of Rep78 to Rep52 in the (insect) cell. Preferably, the combination of first and second expression cassettes in the cell produces a molar ratio of Rep78 to Rep52 in the range of 1:10 to 10:1, 1:5 to 5:1, or 1:3 to 3:1 in the (insect) cell. More preferably, the first nucleic acid construct produces a molar ratio of Rep78 to Rep52 that is at least 1:2, 1:3, 1:5 or 1:10. The molar ration of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

A desired molar ratio of Rep78 to Rep52 can be obtained by the choice of the promoters in respectively the first and second expression cassettes as herein further described above. Alternatively or in combination, the desired molar ratio of Rep78 to Rep52 can be obtained by using means to reduce the steady state level of the at least one of parvoviral Rep 78 and 68 proteins.

Thus, in one embodiment of the method of the invention, the nucleotide sequence encoding the mRNA for the at least one of parvoviral Rep 78 and 68 proteins comprises a modification that affects a reduced steady state level of the at least one of parvoviral Rep 78 and 68 proteins. The reduced steady state condition can be achieved for example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998.

In a preferred embodiment, the nucleotide sequence encoding at least one of parvoviral Rep78 and 68 proteins comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding at least one of parvoviral Rep78 and 68 proteins comprises an open reading frame that starts with a suboptimal translation initiation codon and has no initiation codons further downstream. The suboptimal initiation codon preferably affects partial exon skipping upon expression of the nucleotide sequence in an insect cell. Preferably, the suboptimal initiation codon affects partial exon skipping in an insect cell so as to produce in the insect cell a molar ratio of Rep78 to Rep52 in the range of 1:10 to 10:1, 1:5 to 5:1, or 1:3 to 3:1. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

In one embodiment of the method of the invention, in step e) the expression of the parvoviral Rep 78 and 52 proteins is induced by introducing into the cells of the single cell clone a first nucleotide sequence comprising an expression cassette for expression of the a baculoviral immediate-early protein (IE1) or its splice variant (IE0) transcriptional transregulator, wherein preferably, the first nucleotide sequence is comprised in a baculoviral vector. In a further embodiment of the method of the invention, in step e) simultaneous with the introduction of the first nucleotide sequence a second and a third nucleotide sequence is introduced into the cells of the single cell clone, wherein a) a second nucleotide sequence comprises parvoviral capsid protein coding sequences operably linked to a third promoter for expression in the insect cell; and b) a third nucleotide sequence comprising a transgene that is flanked by at least one parvoviral inverted terminal repeat sequence, wherein preferably, the second and third nucleotide sequences are comprised in at least one baculoviral vector, wherein more preferably the first, second and third nucleotide sequences are comprised together in a single baculoviral vector, and wherein optionally, in step e) and the production parvoviral particles is detected, wherein a single cell clone wherein production of parvoviral particles is detected is selected.

### Capsid proteins

For production of a complete parvoviral gene therapy vector virion, a single cell clone obtained/obtainable in a method of the invention can be transformed with a further (third) expression cassette comprising a nucleotide sequence comprising parvoviral capsid protein coding sequences operably linked to a third promoter for expression in the insect cell.

A nucleotide sequence encoding a parvoviral capsid (Cap) protein is herein understood to comprise nucleotide sequences encoding one or more of the three parvoviral capsid proteins, VP1, -2 and -3. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 7, 8, 9, 10, 11, 12 or 13) or primates (e.g., serotypes 1 and 4), of which the nucleotide and amino acid sequences are listed in Lubelski et al. US2017356008, which is incorporated herein in its entirety by reference. Hence, the nucleic acid construct according to the present invention can comprise an entire open reading frame for AAV capsid proteins as disclosed by Lubelski et al. US2017356008. Alternatively, the sequence can be man-made, for example, the sequence may be a hybrid form or may be codon optimized, such as for example by codon usage of AcmNPv or Spodoptera frugiperda. For example, the capsid sequence may be composed of the VP2 and VP3 sequences of AAV1 whereas the remainder of the VP1 sequence is of AAV5. A preferred capsid protein is AAV5 or an AAV2/5 hybrid, preferably (SEQ ID NO. 4 and 3, respectively in this application) or AAV8, preferably SEQ ID NO. 13 (see SEQ ID NO. 28 in Lubelski et al. US2017356008). Thus, in a preferred embodiment, the AAV capsid proteins are AAV serotype 5, hybrid serotype 2/5 or AAV serotype 8 capsid proteins that have been modified according to the invention. More preferably, the AAV capsid proteins are AAV serotype 5 capsid proteins that have been modified according to the invention. More preferably, the cap-coding sequences are at least CAP AAV2/5 (SEQ ID NO. 3) and AAV5 (SEQ ID NO. 4). It is understood that the exact molecular weights of the capsid proteins, as well as the exact positions of the translation initiation codons may differ between different parvoviruses. However, the skilled person will know how to identify the corresponding position in nucleotide sequence from other parvoviruses than AAV5. Alternatively, the sequence encoding AAV capsid proteins is a man-made sequence, for example as a result of directed evolution experiments. This can include generation of capsid libraries via DNA shuffling, error prone PCR, bioinformatics rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; September 1-5, 2004, pages 3520-3523; Asuri et al., 2012, Molecular Therapy 20(2):329-3389; Lisowski et al., 2014, Nature 506(7488):382-386, herein incorporated by reference.

In a preferred embodiment of the invention, the open reading frame encoding a VP1 capsid protein starts with non-canonical translation initiation codon selected from the group consisting of: ACG, ATT, ATA, AGA, AGG, AAA, CTG, CTT, CTC, CTA, CGA, CGC, TTG, TAG and GTG. Preferably, the non-canonical translation initiation codon is selected from the group consisting of GTG, CTG, ACG, and TTG, more preferably the non-canonical translation initiation codon is CTG.

The nucleotide sequence of the invention for expression of the AAV capsid proteins further preferably comprises at least one modification of the nucleotide sequence encoding AAV VP1 capsid protein selected from among a G at nucleotide position 12, an A at nucleotide position 21, and a C at nucleotide position 24 of the VP1 open reading frame, wherein the nucleotide positions correspond to the nucleotide positions of the wild-type nucleotide sequences. A “potential/possible false start site” or “potential/possible false translation initiation codon” is herein understood to mean an in-frame ATG codon located in the coding sequence of the capsid protein(s). Elimination of possible false start sites for translation within the VP1 coding sequences of other serotypes will be well understood by an artisan of skill in the art, as will be the elimination of putative splice sites that may be recognized in insect cells. For example, the modification of the nucleotide at position 12 is not required for recombinant AAV5, since the nucleotide T is not giving rise to a false ATG codon. Specific examples of a nucleotide sequence encoding parvovirus capsid proteins are given in SEQ ID NO. 16, 17 and 18. Nucleotide sequences encoding parvoviral Cap and/or Rep proteins of the invention may also be defined by their capability to hybridise with the nucleotide sequences of SEQ ID NO. 16, 17, 18 and 5 to 9, respectively, under moderate, or preferably under stringent hybridisation conditions.

The capsid protein coding sequences may be present in various forms. E.g. separate coding sequences for each of the capsid proteins VP1, -2 and -3 may be used, whereby each coding sequence is operably linked to expression control sequences for expression in an insect cell. More preferably, however, the third expression cassette comprises a nucleotide sequence comprising a single open reading frame encoding all three of the parvoviral (AAV) VP1, VP2, and VP3 capsid proteins, wherein the initiation codon for translation of the VP1 capsid protein is a suboptimal initiation codon that is not ATG as e.g. described by Urabe et al., (2002, supra) and in WO2007/046703. A suboptimal initiation codon for the VP1 capsid protein may be as defined above for the Rep78 protein. More preferred suboptimal initiation codons for the VP1 capsid protein may be selected from ACG, TTG, CTG and GTG, of which CTG and GTG are most preferred.

In an alternative embodiment, the second expression cassette comprises a nucleotide sequence comprising a single open reading frame encoding all three of the parvoviral (AAV) VP1, VP2, and VP3 capsid proteins, wherein the initiation codon for translation of the VP1 capsid protein is ATG and wherein the mRNA coding for the VP1 capsid protein as encoded in the nucleotide sequence comprises an alternative start codon which is out of frame with the open reading frame the VP1 capsid protein (as described in WO2019/016349). Preferably, the alternative start codon is selected from the group consisting of CTG, ATG, ACG, TTG, GTG, CTC and CTT, of which ATG is preferred. Preferably, the AAV capsid proteins are AAV5 serotype capsid proteins. Preferably in this embodiment, the nucleotide sequence comprises an alternative open reading frame starting with the alternative start codon that encompasses said ATG translation initiation codon for VP1, whereby preferably, the alternative open reading frame following the alternative start codon encodes a peptide of up to 20 amino acids.

The nucleotide sequence comprised in the second expression cassette for expression of the capsid proteins may further comprise one or more modifications as described in WO2007/046703. Various further modifications of VP coding regions are known to the skilled artisan which could either increase yield of VP and virion or have other desired effects, such as altered tropism or reduce antigenicity of the virion. These modifications are within the scope of the present invention.

In one embodiment, the expression of VP1 is increased as compared to the expression of VP2 and VP3. VP1 expression may be increased by supplementation of VP1, by introduction into the insect cell of a single vector comprising nucleotide sequences for the VP1 as has been described in WO 2007/084773.

Typically, in a method of the invention, at least one open reading frame comprising nucleotide sequences encoding the VP1, VP2 and VP3 capsid proteins or at least one open reading frame, comprising an open reading frame comprising nucleotide sequences encoding at least one of the Rep78 and Rep68 proteins. In one embodiment, the VP1, VP2 and VP3 capsid proteins or at least one open reading frame comprising an open reading frame comprising nucleotide sequences encoding at least one of the Rep78 and Rep68 proteins does not comprise an artificial intron (or a sequence derived from an artificial intron). That is to say, at least open reading frames used to encode Rep or VP proteins will not comprise an artificial intron. By artificial intron is meant to be an intron which would not naturally occur in an adeno-associated virus Rep or Cap sequence, for example an intron which has been engineered so as to permit functional splicing within an insect cell. An artificial intron in this context therefore encompass wild-type insect cell introns. An expression cassette of the invention may comprise native truncated intron sequence (by native is meant sequence naturally occurring in an adeno-associated virus)—such sequences are not intended to fall within the meaning of artificial intron as defined herein.

In the invention, one possibility is that no open reading frame comprising nucleotide sequences encoding the VP1, VP2 and VP3 capsid proteins and/or no open reading frame comprising nucleotide sequences encoding at least one of the Rep78 and Rep68 proteins comprises an artificial intron.

Preferably the nucleotide sequence of the invention encoding the AAV capsid proteins is operably linked to expression control sequences for expression in an insect cell. These expression control sequences will at least include a promoter that is active in insect cells. A suitable promoter for transcription of the nucleotide sequence of the invention encoding of the AAV capsid proteins is e.g. the polyhedron promoter (polH), such a polH promoter SEQ ID NO, 14 and shortened version thereof SEQ ID NO. 15 (see SEQ ID NO.:53 and shortened version thereof SEQ ID NO. 54 in Lubelski et al. US2017356008). However, other promoters that are active in insect cells and that may be selected according to the invention are known in the art, e.g. a polyhedrin (polH) promoter, p10 promoter, p35 promoter, 4xHsp27 EcRE+minimal Hsp70 promoter, deltaE1 promoter, E1 promoter or IE-1 promoter and further promoters described in the above references.

### Viral Vectors

The present invention relates to methods for producing a single insect cell clone that can be used for the production of parvoviruses, in particular dependoviruses such as infectious human or simian AAV, and the components thereof (e.g., a parvovirus genome) for use as vectors for introduction and/or expression of nucleic acids in mammalian cells, preferably human cells.

A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wildtype (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. wtAAV infection in mammalian cells relies for the capsid proteins production on a combination of alternate usage of two splice acceptor sites and the suboptimal utilization of an ACG initiation codon for VP2.

A “recombinant parvoviral or AAV vector” (or “rAAV vector”) herein refers to a vector comprising one or more polynucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

The invention relates to a method for producing a recombinant parvoviral (rAAV) virion, comprising a recombinant parvoviral (rAAV) vector, in an insect cell. In one embodiment, the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV). Preferably, the nucleotide sequences are of the same serotype. More preferably, the nucleotide sequences differ from each other in that they may be either codon optimized, AT-optimized or GC-optimized, to minimize or prevent recombination. Preferably, the difference in the first and the second nucleotide sequence coding for the common amino acid sequences of a parvoviral Rep protein is maximised (i.e. the nucleotide identity is minimised) by one or more of: a) changing the codon bias of the first nucleotide sequence coding for the parvoviral Rep common amino acid sequence; b) changing the codon bias of the second nucleotide sequence coding for the parvoviral Rep common amino acid sequence; c) changing the GC-content of the first nucleotide sequence coding for the common amino acid sequence; and d) changing the GC-content of the second nucleotide sequence coding for the common amino acid sequence. Codon optimisation may be performed on the basis of the codon usage of the insect cell used in the method of the invention, preferably Spodoptera frugiperda, as may be found in a codon usage database (see e.g. http://www.kazusa.or.jp/codon/). Suitable computer programs for codon optimisation are available to the skilled person (see e.g. Jayaraj et al., 2005, Nucl. Acids Res. 33(9):3011-3016; and on the internet). Alternatively, the optimisations can be done by hand, using the same codon usage database.

### Transgene

For production of a complete parvoviral gene therapy vector virion, a single cell clone obtained/obtainable in a method of the invention can be further transformed with: a) a nucleotide sequence comprising parvoviral capsid protein coding sequences operably linked to a third promoter for expression in the insect cell; b) a nucleotide sequence comprising a transgene that is flanked by at least one parvoviral inverted terminal repeat sequence; and, c) a nucleotide sequence comprising an expression cassette for expression of the transcriptional transregulator.

In a further embodiment, the nucleotide sequences of at least one of a) and b) is comprised in a baculoviral vector, wherein, preferably the nucleotide sequences of at least one of a), b) and c) are comprised in the baculoviral vector comprising the expression cassette for expression of the transcriptional transregulator.

In the context of the invention “at least one parvoviral inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition site for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. More preferably AAV2 ITRs are used. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between. In a preferred embodiment a nucleotide sequence of (i) is flanked on either side by parvoviral inverted terminal repeat nucleotide sequences.

In the embodiments of the invention, the nucleotide sequence comprising the transgene (encoding either a gene product of interest or comprising a nucleotide sequence targeting a gene of interest) that is flanked by at least one parvoviral ITR sequence preferably becomes incorporated into the genome of a recombinant parvoviral (rAAV) vector produced in the insect cell. Preferably, the transgene encodes a gene product of interest for expression in a mammalian cell. Preferably, the transgene comprises at least one nucleotide sequence targeting a gene of interest for silencing said gene of interest in a mammalian cell. Preferably, the nucleotide sequence comprising the transgene is flanked by two parvoviral (AAV) ITR nucleotide sequences and wherein the transgene is located in between the two parvoviral (AAV) ITR nucleotide sequences. Preferably, the nucleotide sequence encoding a gene product of interest (for expression in the mammalian cell) or comprising a nucleotide sequence targeting a gene of interest (for silencing the gene of interest in the mammalian cell) will be incorporated into the recombinant parvoviral (rAAV) vector produced in the insect cell if it is located between two regular ITRs, or is located on either side of an ITR engineered with two D regions.

AAV sequences that may be used in the present invention for the production of a recombinant AAV virion in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8. AAV9, AAV10, AAV11, AAV12 or AAV13 or newly developed AAV-like particles obtained by e.g. capsid shuffling techniques and AAV capsid libraries, or from newly and synthetically designed, developed or evolved capsid, such as the Anc-80 capsid.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV capsid proteins, also known as VP proteins, are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped rAAV particles comprising the capsid proteins of a serotype (e.g., AAV3) and the Rep and/or ITR sequences of another AAV serotype (e.g., AAV2). Such pseudotyped rAAV particles are a part of the present invention.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR, Rep, or VP can be used in place of wild-type AAV ITR, Rep, or VP sequences.

In a preferred embodiment, an insect cell of the invention further comprises a nucleotide sequence comprising a transgene that is flanked by at least one parvoviral ITR sequence. Thus, preferably the nucleotide sequence comprises at least one AAV ITR and at least one nucleotide sequence encoding a gene product of interest (preferably for expression in a mammalian cell) or a nucleotide sequence targeting a gene of interest (preferably for silencing said gene of interest in a mammalian cell), whereby preferably the at least one nucleotide sequence encoding a gene product of interest or targeting a gene of interest becomes incorporated into the genome of an AAV produced in the insect cell. Preferably, the at least one nucleotide sequence encoding a gene product of interest is a sequence for expression in a mammalian cell. Preferably, the at least one nucleotide sequence targeting a gene of interest is a sequence for silencing said gene of interest in a mammalian cell Preferably, the nucleotide sequence comprises two AAV ITR nucleotide sequences and wherein the at least one nucleotide sequence encoding a gene product of interest or targeting a gene of interest is located between the two AAV ITR nucleotide sequences. Preferably, the nucleotide sequence encoding a gene product of interest (for expression in the mammalian cell) or the nucleotide sequence targeting a gene of interest (for silencing said gene of interest in a mammalian cell) will be incorporated into the AAV genome produced in the insect cell if it is located between two regular ITRs, or is located on either side of an ITR engineered with two D regions. Thus, in a preferred embodiment, the invention provides an insect cell according to the invention, wherein the nucleotide sequence comprises two AAV ITR nucleotide sequences and wherein the at least one nucleotide sequence encoding a gene product of interest or the at least one nucleotide sequence targeting a gene of interest is located between the two AAV ITR nucleotide sequences.

Typically, the transgene, including ITRs and promoter & polyadenylation sequences, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

The nucleotide sequence comprising the transgene as defined herein above may thus comprise a nucleotide sequence encoding a gene product of interest (for expression in the mammalian cell) or the nucleotide sequence targeting a gene of interest (for silencing said gene of interest in a mammalian cell), and may be located such that it will be incorporated into an recombinant parvoviral (rAAV) vector replicated in the insect cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant parvoviral (rAAV) vector produced in accordance with the present invention. The nucleotide sequence may e.g. encode a protein or it may express an RNAi agent, i.e. an RNA molecule that is capable of RNA interference such as, e.g. an shRNA (short hairpinRNA) or an siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in the insect cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. silencing a toxic gain-of-function variant), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidemia or amyotrophic lateral sclerosis (ALS).

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E.coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression (as disclosed in PCT/EP2019/081743) a promoter may be selected from an a1-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10).

Various modifications of the nucleotide sequences as defined above, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

### AAV

In the recombinant parvoviral (rAAV) vectors of the invention the at least one nucleotide sequence(s) encoding a transgene or gene product of interest for expression in a mammalian cell, preferably is/are operably linked to at least one mammalian cell-compatible expression control sequence, e.g., a promoter. As discussed in the above, many such promoters are known in the art.

AAV is able to infect a number of mammalian cells. See, e.g., Tratschin et al. (1985, Mol. Cell Biol. 5:3251-3260) and Grimm et al. (1999, Hum. Gene Ther. 10:2445-2450). However, AAV transduction of human synovial fibroblasts is significantly more efficient than in similar murine cells, Jennings et al., Arthritis Res, 3:1 (2001), and the cellular tropicity of AAV differs among serotypes. See, e.g., Davidson et al. (2000, Proc. Natl. Acad. Sci. USA, 97:3428-3432), who discuss differences among AAV2, AAV4, and AAV5 with respect to mammalian CNS cell tropism and transduction efficiency. In a preferred embodiment, a host cell of the invention is any mammalian cell that may be infected by a parvoviral virion, for example, but not limited to, a muscle cell, a liver cell, a nerve cell, a glial cell and an epithelial cell. In a preferred embodiment a host cell of the invention is a human cell.

### Cell Bank

In a further aspect, the invention relates to a method for producing a cell bank of a single cell clone of an insect cell comprising integrated into the genome of the cell at least one expression cassette for inducible expression of parvoviral Rep 78 and 52 proteins, the method comprising the steps of: a) producing and/or selecting a single cell clone in a method according to the invention; b) expanding the single cell clone obtained in a); and, c) distributing the expanded single cell clone obtained in b) over a plurality of vials.

In one embodiment, the method for producing a cell bank also include storage of the vials.

In a further aspect, the invention relates to a single cell clone obtained, obtainable, produced and/or selected in method according to the invention, or a cell bank consisting of a plurality of vials of the single cell clone produced in a method according to the invention. In one embodiment, the single cell clone is a single cell clone of an insect cell as herein defined above and obtained or obtainable in method according to the invention. In a preferred embodiment, the single cell clone is a single cell clone of an insect cell for producing a parvoviral vector, wherein the insect cell has been modified to comprise at least one expression cassette for expression of parvoviral Rep 78 and 52 proteins as herein defined above. Preferably, the insect cell has been modified to comprise at least one expression cassette for inducible expression of at least one of parvoviral Rep 78 and 52 proteins as herein defined above.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. Flow chart for clone outgrowth stage, intermediate mirroring stage and clone expansion stage.

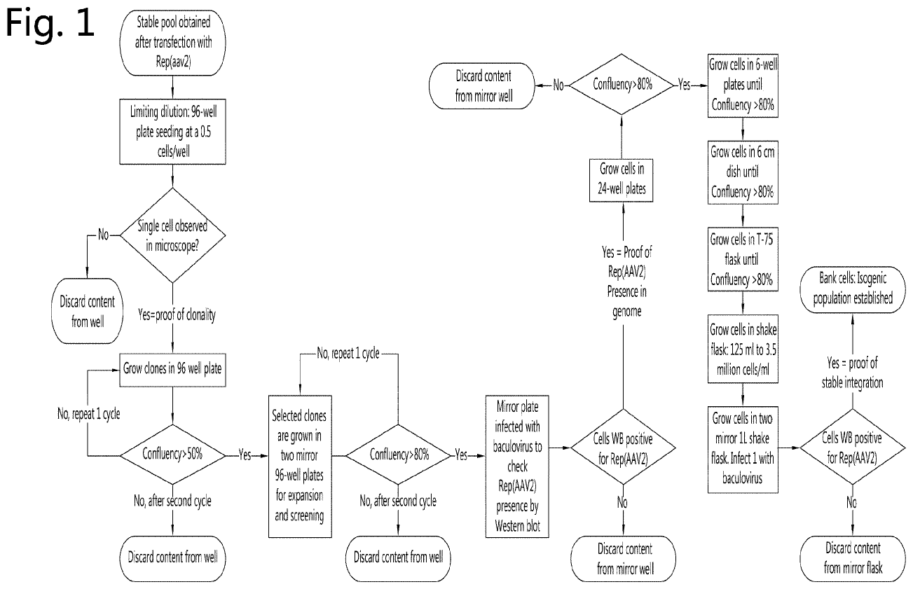

FIG. 2. Post-seeding pictures of limiting dilutions of iRep cells, taken using the cell metric software in order to identify which well had a single cell clone in the plates.

### EXAMPLES

### Solutions

Dilute the Blasticidin S hydrochloride 25 mg into 2.5 ml WFI to get a final concentration of 10 mg/ml. Filter the diluted Blasticidin S hydrochloride 10 mg/ml using a syringe Filter Unit, 0.22 μm, PVDF and a BD syringe 5 ml. The filtered Blasticidin S hydrochloride 10 mg/ml is ready to use. Glutamine 200 mM is commercially available (Thermo Scientific, Catalogue Number: 25030149) Express Sf+ is a cell line derived from SF9 described in Smith et al., 2000 (WO 00/20561). iREP refers to the stable cell pool with integrated Rep cassette from AAV2.

A method to generate a single insect cell (Expres Sf+) clone from a cells suspension. After transfection, a mixed-population of genetically different cells has been obtained, also known as a stable pool. Since the transgene can exist in different numbers of copies and can be integrated at different location in the genome, the cell pool contains cells with different levels of transgene expression and differences in growth performance. In order to obtain a monoclonal homogeneous cell line, single cell clones were obtained by using limiting dilution. The single cell clone isolation was confirmed and verified by using the VIPS™ (verified in-situ plate seeding) system including the cell metric software.

### Example 1

Day −8: the iRep cells (the construction of which is described in WO 2021/198510) and SF+cells are thawed by hand for 15 minutes and back diluting into 25-ml fresh Sf-900™ II (Gibco™, ThermoFisher Scientific). The cell viability and density are measured and recorded according to the manufacturer instruction of the cell counter device.

### Seed the iRep and the SF+Cells

The cell culture is diluted with the new medium for same volume passaging (i.e. 50 mL to 50 ml) to achieve a viable cell density of 1.5×10⁶ Viable Cells/mL at final concentration in a 125 mL shake flask. The cells require fresh medium exchange approximately every 2 to 3 days always back to diluting between 0.5×10⁶ Viable Cells/mL and 1.0×10⁶ Viable Cells/mL.

Day −6: iRep and SF+cells seeding started with warming up the Sf900 II media at 28° C. and 1 hour. The cell viability and density was measured according to the manufacturer instruction of cell counter device.

### Seed the iRep Cells

50 ml of iRep cells were prepared at a final viable cell density of 5×10⁵ Viable Cells/mL in a 125-mL shaker flask. 100 uL of Blasticidin 10 mg/mL was added into 50 mL of cells suspension to result in a final concentration of 20 ug/mL Blasticidin, which was used as a cell selection pressure. The cells were grown in a 28° C. shaker incubator at 135 rpm for 72 hours.

### Seed SF+Cells

100 mL of SF+cells were prepared at a final viable cell density of 5×10⁵ Viable Cells/mL in a 250 ml shaker flask. The cells required fresh medium approximately every 2 to 3 days according to always back to diluting between 0.5×10⁶ Viable Cells/mL and 1.0×10⁶ Viable Cells/mL. The cells were grown in a shaker incubator at 28° C. and 135 rpm for 72 hours.

Day −3: iRep and SF+cells seeding started with warming up the Sf900 II media at 28° C. and 1 hour. The cells viability and density was checked according to the manufacturer instruction of cell counter device.

### Seed the iRep Cells

50 mL of iRep cells were prepared at a final viable cell density of 1×10⁶ Viable Cells/mL in a 125-mL shaker flask. 100 uL of Blasticidin 10 mg/mL into 50 mL of cells suspension to result in a final concentration of 20 ug/mL Blasticidin, which was used as a cell selection pressure. The cells were grown in a 28° C. shaker incubator at 135 rpm for 48 hours.

### Seed the SF+Cells

200 ml of SF+cells at a final viable cell density of 1×10⁶ Viable Cells/mL in a 500-mL shaker flask was prepared. The cells were grown in a 28° C. shaker incubator at 135 rpm for 24 hours.

Day-2: SF+cells seeding for spent media preparation started warming up the Sf900 II media at 28° C. and 1 hour. The cells viability and density was checked according to the manufacturer instruction of cell counter device. 400 mL of SF+cells were prepared at a final viable cell density of 5×10⁵ Viable Cells/mL in a 1 L shaker flask. The cells were grown in a 28° C. shaker incubator at 135 rpm for 48 hours.

Day −1: iRep cells seeding started with warming up the Sf900 II media at 28° C. and 1 hour. The cells viability and density was checked according to the manufacturer instruction of cell counter device. Viable cell density after incubation should be preferably between 2.5−6×10⁶ Viable Cells/mL with >90% viability. 50 mL of iRep cells were prepared at a final viable cell density of 1×10⁶ Viable Cells/mL in a 125-mL shaker flask. Blasticidin pressure was not added. The cells were grown in a 28° C. shaker incubator at 135 rpm for 48 hours.

Day 0: Limiting dilution and 96-well plates seeding started with warming up the Sf900 II media at 28° C. and 1 hour. The cells viability (should be >98%) and density was checked according to the manufacturer instruction of cell counter device less than 16 hours after splitting for the iRep cells.

### Spent Media Preparation

Inside a biosafety cabinet to keep aseptic conditions, 400 mL of SF+cells suspension was divided into 50-mL Falcon tubes and centrifuged at 1900 g for 15 minutes at 4° C. The supernatants were filtered with a filter unit 0.22 um by using a syringe. The filtrate was collected with a new sterile 50-mL Falcon tube.

### Limiting Dilution Conditions Preparation

250 mL condition medium was composed of 85% (v/v) spent medium, 10% (v/v) AuFBS, 5% (v/v) 200 mM L-glutamine was prepared. 36 mL condition medium was pipetted into three separate sterile 50 mL Falcon tubes (Falcon tubes 2, 3 and 4 according to Table 1) and 45 mL condition medium was pipetted into two separate sterile 50 mL tubes (Falcon tubes 5 and 6, according to Table 1) for limiting dilution.

### Limiting dilution

40 mL iRep culture in a 50-mL Falcon (Falcon tube 1 according to Table 1) with the condition medium to get a final cell concentration of 2.5×10⁴ Viable Cells/mL was prepared and vortexed at maximum speed for a few seconds at room temperature to mix well before proceeding the next step. To perform the series of 10-fold dilution, the following sequential dilutions were used to achieve the desire cell density (Error! Reference source not found.), and each resulting dilution was vortexed before proceeding the next dilution step:

For the dilution from Falcon 1 to 4, 4 mL of the previous dilution was pipetted into 36 mL of the next dilution in each step. For the dilution from Falcon 4 to Falcons 5a and 5b (according to Table 1), 5 mL of the previous dilution was pipetted into 45 mL of the next dilution in each step.

TABLE 1
Dilution for each Falcon tube

| | | | Number of cells per |
|---|---|---|---|
| | | | well after 96-well |
| | Total dilution from | Desired cell density | plate seeding |
| Falcon | Falcon 1 | (cells/mL) | (cells/200 uL) |
| 1 | 0x | 25000 | 5000 |
| 2 | 10x | 2500 | 500 |
| 3 | 100x | 250 | 50 |
| 4 | 1000x | 25 | 5 |
| 5a | 10000x | 2.5 | 0.5 |
| 5b | 10000x | 2.5 | 0.5 |

Perform the 96-well plates seeding for iRep cells as follows

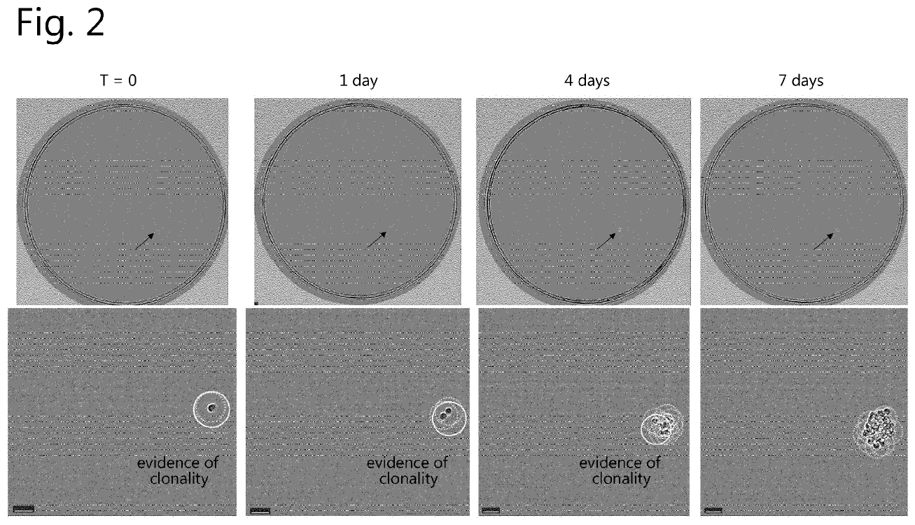

Cultures from Falcon 1-4 were used as reference for the growth of the stable pool. 200 uL of culture from Falcon 1-4 was pipetted into each well of a 96-well plate where the A1 well of the 96-well plates from Falcon 2-4 was filled with culture from Falcon 1, which is the overseeded positive control. 200 uL of culture from Falcon 5a and 5b was pipetted into each well of five separate 96-well plates where the A1 well is filled with the culture from Falcon 1 which is the overseeded positive control. The cells on all 96-well plates were grown in a 28° C. static incubator, with 1% CO2 for 2 hours. Post-seeding pictures were taken using the cell metric software in order to identify which well has a single cell clone in the plates and the following steps were carried out with those single cell clones (FIG. 2). Only single intact cells should be considered and marked clearly in the monitoring software, other wells can be discarded after passaging. The cells on all 96-well plates were grown in a static incubator at 28° C. with fixed 1% CO2 for 1 week.

Day 7: Media change for iRep seeding in 96-well plates started with warming up the Sf900 II media at 28° C. for 1 h. The growth performance of the 96-well plates obtained from Falcon1-4 was compared by evaluating the confluency of the wells. If the general confluency of each plate was as according to the series of dilution made (Error! Reference source not found.), this ensured the performance of the limited dilution. The reference plates were discarded after evaluation. 100 uL culture was removed out of each well from the 96-well plates. 100 ul of fresh Sf900 II media was added into each well of the 96-well plates. 1 week post-seeding pictures were taken of the 96-well plates using the cell metric software. The cells were grown in a static incubator at 28° C. with fixed 1% CO2. The above mentioned steps were repeated with the culture in 96-well plate every 7 days for in total 2 cycles.

Day 28: Cell passages in 96-well plates for continuous culture and Western blot analysis. The sf900ii media was warmed and pictures of the 96-well plate were taken using the cell metric. In the well with the approval of clonality, the single cell got divided and at this stage it should have a confluency >50% in the well of the 96-well plate.

Two new 96-well plates were prepared by adding 100 uL of Sf900 II media into each well. The plates were prepared for continuous culture and the analysis of Rep genes expression using Western blot. 100 uL culture of the isolated clone which has a confluency >50% in the 96-well plate was transferred to two separate 96-well plates and gently aspirated up and down several times (30×) to perfectly resuspend and disperse the colony before transfer. The cells were grown in a static incubator at 28 ° C. and with fixed 1% CO2.

Day 31: Cell seeding in 96-well plate for Western Blot analysis started by checking under the microscope if the cell seeding in the 96-well plate had reached >50% confluency and directly proceed to the next steps. If not reached, the 96-well plates were returned to the incubator until they reach minimum 50% confluency. 100 uL of liquid was removed from the 96-well plate without disturbing the cell layer at the bottom of the well.

### Infection with Baculovirus

The expression of the integrated Rep genes in iRep is coactivated by the promoter of the baculovirus, so proof of the presence of the integrated Rep genes can only be obtained by infection of iRep cells with baculovirus. Therefore, 100 ul of a suspension in Sf900 II media with at least 1×10⁶ infectious particles/mL of a baculovirus bearing Cap-Luciferase insert in its genome was added into each well to infect the iRep cells. For the positive control, 100 uL of ExpresSf+wild type cells were seeded with a final viable cell concentration of 1×10⁶ Viable Cells/mL in a well of the 96-well plate and 100 uL a suspension in Sf900 II media with at least 1×10⁶infectious particles/mL of a baculovirus bearing a Rep(AAV2) insert in its genome was added to infect the parental cell line cells. For the negative control, 100 uL of ExpresSf+wild type cells (1e4 viable cells/mL) was seeded in a well of the 96-well plate and 100 uL of Sf900 II media was added.

Day 33: Samples harvested for Western Blot analysis (Day 1) 96-well are used to demonstrate that the isogenic population established actually contains the gene of interest, i.e. Rep(AAV2). The procedure starts with preparing the cell lysis buffer by dissolving 1 protease inhibitor tablet (Protease inhibitor cocktail complete, mini, EDTA-free protease inhibitor cocktail Sigma #11836170001) in 50 mL RIPA lysis buffer (Sigma #R0278-50ML). The whole spent media was removed from each well in the 96-well plates without disturbing the cell layer at the bottom of the well. 15 uL of cell lysis buffer was added to each well.

1 uL of 10×diluted (in cell lysis buffer) benzonase (Benzonase®, Sigma Aldrich, Activity (DNA; pH8.0; 30 min; 37° C.): 250-350 U/μl) was added and pipetted up and down to mix well. The cell lysate was incubated in the 96-well plate in a static incubator at 37° C. for 30 minutes. 5 uL of 4×Laemmli Sample Buffer containing 10% β-Mercaptoethanol was added. The cell lysate was transferred from each well to a thermocycler 96-well plate and the plate was sealed with a PCR aluminium lid. The plate was incubated for 5 minutes at 95° C. in a thermocycler.

The plate was stored at −80° C. for further analysis or Western Blot analysis was performed where the first (Mouse monoclonal anti-Rep 303.9, Progen, cat. #65169, dilution 1:500) and second (Anti-mouse IgG, HRP-linked Ab, Ref: 04/2020, Lot: 35, 70765, dilution 1:1000) antibodies were used for membrane incubation antibody solution was distributed equally over the PVDF membrane after removing the excess PBS buffer. The PVDF membrane was kept in a close container to avoid evaporation and was followed by incubation at room temperature. 1st antibody solution requires overnight incubation. 2nd antibody solution requires 2 hours incubation. Both solutions were performed individually.

### Cell Seeding Expansion from 96-Well Plate to 24-Well Plate

Cells confluency was checked under the microscope or using the cell metric software to ensure it was >80% before proceeding to next steps. A new 24-well plate was prepared by adding 400 uL of fresh media Sf900 II into each well of the plate. Cell seed were selected with Rep genes expression according to the results of Western blot analysis and gently aspirated up and down several times (30×) to perfectly disperse the resulting monoclonal colony after cell growth from a single cell in the selected well of the 96-well plate and 200 uL of the cell suspension was transferred to each well of the new 24-well plate. The cells were grown in a static incubator at 28 ° C. and with fixed 1% CO2.

### Media Change for Cell Seedings in 24-Well Plate

If the cells confluency in 24-well plate was <80%, the selected clones were not moved to the next step. However, media had to be changed and this step was carried out every 3-4 days. If the cells were interrupted (e.g. splitting the culture in to half) when the confluency was below 80%, the cells may enter into a arrested growth phase and death. To do so, 400 uL of each cell suspension was gently removed in each well of the 24-well plate without disturbing the cell layer at the bottom of the well. 800 uL of fresh Sf900 II media was added into each well of the plate. The cells were grown in a static incubator at 28° C. and with fixed 1% CO2.

If, after 3-4 days cultivation later the cells confluency was still <80%, we proceeded with a new media change as follows: 800 uL of cell suspension was gently removed in each well of the 24-well plate without disturbing the cell layer at the bottom of the well. 800 ul of fresh Sf900 II media was added into each well of the plate. The cells were grown in a static incubator at 28° C. and with fixed 1% CO2.

### Cell Seeding Expansion from 24-Well Plate to 6-Well Plate

A cell confluency of >80% was checked for under the microscope. The clonality was checked on the post seeding picture after seeding from limited dilution.

1. A method for producing a single cell clone of an insect cell comprising at least one expression cassette for inducible expression of parvoviral Rep 78 and 52 proteins integrated into the genome of the cell, the method comprising:
(a) preparing a series of sequential dilutions from a culture comprising a plurality of the insect cell;
(b) seeding an aliquot from a dilution obtained in (a) into at least one culture container at a cell density of less than one insect cell per container;
(c) incubating the culture container under conditions that are conducive to growth of the insect cell and that do not induce expression of the parvoviral Rep 78 and 52 proteins; and,
(d) selecting at least one single cell clone from at least one culture container in which the insect cells have grown.

2. The method according to claim 1, further comprising inspecting the culture container(s), optionally by visual inspection, to identify a culture container comprising no more than a single cell.

3. The method according to claim 1, further comprising:
(e) inducing expression of the parvoviral Rep 78 and 52 proteins in a sample taken from at least one single cell clone obtained in (d), detecting induced expression of the parvoviral Rep 78 and 52 proteins, and selecting at least one single cell clone wherein induced expression of the parvoviral Rep 78 and 52 proteins is detected.

4. A method according to claim 1, wherein the at least one expression cassette for inducible expression of the parvoviral Rep 78 and 52 proteins comprises at least one baculoviral promoter that is operably linked to at least one sequence coding for the parvoviral Rep 78 and 52 proteins, wherein the at least one baculoviral promoter is operably linked to a baculoviral homologous region (hr) enhancer element that is dependent on a baculoviral immediate-early protein (IE1) or its spice variant (IE0) as transcriptional transregulator, and wherein introduction of the baculoviral immediate-early protein (IE1) or its spice variant (IE0) into the cell induces transcription from the at least one baculoviral promoter.

5. The method according to claim 4, wherein integrated into the genome of the cell is:
(i) a first baculoviral promoter operably linked to a nucleotide sequence encoding an mRNA, translation of which in the cell produces at least one of parvoviral Rep 78 and 68 proteins;
(ii) a second baculoviral promoter operably linked to a nucleotide sequence encoding an mRNA, translation of which in the cell produces at least one of parvoviral Rep 52 and 40 proteins; and
(iii) at least one hr enhancer element that is operably linked to the first and second promoters,
wherein preferably the baculovirus is Autographa californica multicapsid nucleopolyhedrovirus.

6. A method according to claim 4, wherein the hr enhancer element comprises at least one copy of the hr 28-mer sequence CTTTACGAGTAGAATTCTACGCGTAAAA and/or at least one copy of a sequence of which at least 20, 21, 22, 23, 24, 25, 26, or 27 nucleotides are identical to sequence CTTTACGAGTAGAATTCTACGCGTAAAA and which binds to a baculoviral IE1 protein, and wherein the hr enhancer element, when operably linked to an expression cassette comprising a reporter gene operably linked to the polH promoter:
(a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and,
(b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element,
wherein preferably the hr enhancer element is selected from the group consisting of hr1, hr3, hr4b and hr5.

7. The method according to claim 4, wherein the first and second promoters are distinct, and wherein the first promoter is a delayed early baculoviral promoter and the second promoter is a late or very late baculovirus promoter.

8. The method according to claim 7, wherein the first promoter is a 39k promoter and the second promoter is selected from the group consisting of the polH, p10, p6.9 and pSel120 promoters.

9. The method according to claim 4, wherein the at least one of parvoviral Rep 52 and 40 proteins and the at least one of parvoviral Rep 78 and 68 proteins have a common amino acid sequence that is at least 90% identical, while the nucleotide sequence encoding the common amino acid sequence in the mRNA for the at least one of parvoviral Rep 52 and 40 proteins has less than 95, 90, 85, 80, 75, 70, 65 or 60% sequence identity with the nucleotide sequence encoding the common the amino acid sequence in the mRNA for the at least one of parvoviral Rep 78 and 68 proteins.

10. The method according to claim 9, wherein the codon usage in the nucleotide sequence encoding the common the amino acid sequence in the mRNA for the at least one of parvoviral Rep 52 and 40 proteins, is adapted to the codon usage bias of the insect cell than codon usage in the nucleotide sequence encoding the common the amino acid sequence in the mRNA for the at least one of parvoviral Rep 78 and 68 proteins.

11. The method according to claim 4, wherein the nucleotide sequence encoding the mRNA for the at least one of parvoviral Rep 78 and 68 proteins comprises a modification that affects a reduced steady state level of the at least one of parvoviral Rep 78 and 68 proteins.

12. The method according to claim 11, wherein the at least one of parvoviral Rep78 and 68 proteins comprises an open reading frame that starts with a suboptimal translation initiation codon.

13. The method according to claim 12, wherein the suboptimal translation initiation codon is selected from ACG, CTG, TTG, GTG and ATT.

14. The method according to claim 4, wherein the first and second promoters are integrated in the cell's genome in opposite directions of transcription and wherein the at least one enhancer element is present in between the first and second promoters.

15. The method according to claim 14, wherein two enhancer elements are present in between the first and second promoters.

16. The method according to claim 4, wherein expression of the parvoviral Rep 78 and 52 proteins is induced by introducing into the cells of the single cell clone a first nucleotide sequence comprising an expression cassette for expression of the a baculoviral immediate-early protein (IE1) or its spice variant (IE0) transcriptional transregulator.

17. The method according to claim 16, wherein the first nucleotide sequence is comprised in a baculoviral vector.

18. The method according to claim 16, wherein simultaneous with the introduction of the first nucleotide sequence a second and a third nucleotide sequence is introduced into the cells of the single cell clone, wherein
(a) a second nucleotide sequence comprises parvoviral capsid protein coding sequences operably linked to a third promoter for expression in the insect cell; and
(b) a third nucleotide sequence comprising a transgene that is flanked by at least one parvoviral inverted terminal repeat sequence.

19. The method according to claim 18, wherein the second and third nucleotide sequences are comprised in at least one baculoviral vector.

20. The method according to claim 1, wherein:
(a) the insect cell is an insect cell selected from the group consisting of ExpresSf+TM, Sf9, Sf21, TN-368, BTITN-5BI-4 and High-Five™; and/or,
(b) the conditioned medium comprises spent insect cell medium, wherein preferably the spent medium is spent by an insect cell that is an untransformed predecessor of the insect cell, and wherein more preferably the conditioned medium is supplemented with at least one of fetal bovine serum and L-glutamine.

21. A method for producing a cell bank of a single cell clone of an insect cell comprising integrated into the genome of the cell at least one expression cassette for inducible expression of parvoviral Rep 78 and 52 proteins, the method comprising:
(a) producing and/or selecting a single cell clone in a method according to claim 1;
(b) expanding the single cell clone obtained in (a); and,
(c) distributing the expanded single cell clone obtained in (b) over a plurality of vials, and optionally, storing the vials.

22. A single cell clone produced in a method according to claim 1.


SEQUENCE LISTING (18 sequences)

SEQ ID NO: 1 - DNA, 28 bp, synthetic construct
  misc_feature 1..28: note = hr 28-mer
  source 1..28: mol_type = other DNA
ctttacgagtagaattctacgcgtaaaa

SEQ ID NO: 2 - DNA, 1194 bp, synthetic construct
  misc_feature 1..1194: note = Extreme codon optimized Rep52
  source 1..1194: mol_type = other DNA
atggaattggtgggttggcttgtcgataaaggcataacgagcgaaaaacaatggattcaagaagatcaagcatcctatattagctttaacgctgcgagtaatagcagaagccagataaaagcagcgttggacaacgctggtaagatcatgtcgcttacaaagacggcgccggactatctcgtcggtcaacaaccagttgaggacataagttcaaacagaatttacaagatcttggagctcaatggttatgaccctcagtacgctgcgtctgtattcctcggatgggctacaaagaaatttggtaaacgcaatactatttggctcttcggtccagccacaaccggcaaaactaatattgccgaagctattgctcataccgtcccattttatggttgtgtgaattggactaacgaaaatttcccttttaatgattgcgtggataaaatggtcatatggtgggaagaaggtaaaatgacagctaaagtggttgaatccgcgaaggcaatcctgggtggttccaaagtccgtgtcgatcaaaagtgtaaaagctccgctcaaatcgatcctaccccggtaattgttacgagtaatactaatatgtgcgcagttattgacggtaactcgacgacgtttgaacatcaacaacccctccaggatagaatgtttaagtttgagctgactagacgcctcgaccacgatttcggtaaagttactaaacaggaggtgaaggatttctttcgctgggctaaagaccatgtcgtagaagtcgagcacgagttttatgttaagaaaggaggtgctaaaaagcgtcctgctcctagcgatgctgacatctccgaacctaagcgtgtccgtgaaagcgtcgcacaacctagcaccagcgatgctgaggcctccattaattatgctgatcgttatcagaataagtgcagccgccatgtcggtatgaacctcatgctcttcccttgtcgtcagtgtgaacgtatgaaccaaaactcaaacatatgttttacccatggtcaaaaggactgcctggagtgcttccctgtaagtgaatcgcaacctgtaagtgtggtaaagaaagcgtaccaaaagctctgttatatccaccacattatgggcaaagtaccggatgcctgtaccgcgtgtgaccttgtgaacgttgacctggacgattgtatattcgagcagtaa

SEQ ID NO: 3 - DNA, 14265 bp, synthetic construct
  misc_feature 1..14265: note = pVD-polH-Cap-ITR-transgene-ITR or pVD1454cl35 or Bac polH Cap (2/5) Trans (sNano-Luc)
  source 1..14265: mol_type = other DNA
tggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcaggggggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcttcggtcgacggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagaattacgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagactggggcagggaaggcagcaggcaacaccaggagcaggcccagggagaaggcaactggaccgaaggcgcttgtggagaaggagttcatggtggctttaccaacagtaccggattgccaagcttggccgccgaggcccactgactgcgttagcaatttaactgtgataaactaccgcaataaagcttctagtgatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgccaacatggcggtcatattggacatgagccaatataaatgtacatattatgatatagatacaacgtatgcaatggccaatagccaatattgatttatgctatataaccaatgaataatatggctaatggccaatattgaggccagttagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgtgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaaaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctg

SEQ ID NO: 4 - DNA, 2178 bp, synthetic construct
  misc_feature 1..2178: note = Cap AAV2/5 VD92
  source 1..2178: mol_type = other DNA
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 5 - DNA, 1194 bp, synthetic construct
  misc_feature 1..1194: note = Artificial Sequence
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 6 - DNA, 1194 bp, synthetic construct
  misc_feature 1..1194: note = Artificial Sequence
  source 1..1194: mol_type = other DNA
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcaggaggtgaaggacttcttccgttgggctaaggaccacgtggtggaggtggagcacgagttctacgtgaagaagggtggtgctaagaagcgtcctgctccttccgacgctgacatctccgagcctaagcgtgtgcgtgagtccgtggctcagccttccacctccgacgctgaggcttccatcaactacgctgaccgttaccagaacaagtgctcccgtcacgtgggtatgaacctgatgctgttcccttgccgtcagtgcgagcgtatgaaccagaactccaacatctgcttcacccacggtcagaaggactgcctggagtgcttccctgtgtccgagtcccagcctgtgtccgtggtgaagaaggcttaccagaagctgtgctacatccaccacatcatgggtaaggtgcctgacgcttgcaccgcttgcgacctggtgaacgtggacctggacgactgcatcttcgagcagtaa

SEQ ID NO: 7 - DNA, 1194 bp, synthetic construct
  misc_feature 1..1194: note = Artificial Sequence
  source 1..1194: mol_type = other DNA
atggaattagtaggatggttagtagataaaggaataacatcagaaaaacaatggatacaagaagatcaagcatcatatatatcatttaatgcagcatcaaattcaagatcacaaataaaagcagcattagataatgcaggaaaaataatgtcattaacaaaaacagcaccagattatttagtaggacaacaaccagtagaagatatatcatcaaatagaatatataaaatattagaattaaatggatatgatccacaatatgcagcatcagtatttttaggatgggcaacaaaaaaatttggaaaaagaaatacaatatggttatttggaccagcaacaacaggaaaaacaaatatagcagaagcaatagcacatacagtaccattttatggatgtgtaaattggacaaatgaaaattttccatttaatgattgtgtagataaaatggtaatatggtgggaagaaggaaaaatgacagcaaaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 8 - DNA, 1194 bp, synthetic construct
  misc_feature 1..1194: note = Artificial Sequence
  source 1..1194: mol_type = other DNA
atggagctggtggggtggctggtggacaaggggatcacgagcgagaagcagtggatccaggaggaccaggcgagctacatcagcttcaacgcggcgagcaacagccggagccagatcaaggcggcgctggacaacgcggggaagatcatgagcctgacgaagacggcgccggactacctggtggggcagcagccggtggaggacatcagcagcaaccggatctacaagatcctggagctgaacgggtacgacccgcagtacgcggcgagcgtgttcctggggtgggcgacgaagaagttcgggaagcggaacacgatctggctgttcgggccggcgacgacggggaagacgaacatcgcggaggcgatcgcgcacacggtgccgttctacgggtgcgtgaactggacgaacgagaacttcccgttcaacgactgcgtggacaagatggtgatctggtgggaggaggggaagatgacggcgaaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 9 - DNA, 1194 bp, synthetic construct; identical to SEQ ID NO: 5
  misc_feature 1..1194: note = Artificial Sequence
  source 1..1194: mol_type = other DNA

SEQ ID NO: 10 - protein, 397 aa, synthetic construct
  REGION 1..397: note = Artificial Sequence
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 11 - DNA, 1876 bp, synthetic construct
  misc_feature 1..1876: note = Artificial Sequence
  source 1..1876: mol_type = other DNA
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 12 - protein, 621 aa, synthetic construct
  REGION 1..621: note = Artificial Sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 13 - protein, 738 aa, synthetic construct
  REGION 1..738: note = Artificial Sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - DNA, 250 bp, synthetic construct
  misc_feature 1..250: note = polH promoter long
  source 1..250: mol_type = other DNA
tgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagctt

SEQ ID NO: 15 - DNA, 155 bp, synthetic construct
  misc_feature 1..155: note = polH promoter short
  source 1..155: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaat

SEQ ID NO: 16 - DNA, 1800 bp, synthetic construct
  misc_feature 1..1800: note = adeno-associated virus 1; VP2, VP3
  source 1..1800: mol_type = other DNA
acggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 17 - DNA, 1605 bp, synthetic construct
  misc_feature 1..1605: note = adeno-associated virus 1; VP3
  source 1..1605: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 18 - DNA, 1194 bp, synthetic construct; identical to SEQ ID NO: 5
  misc_feature 1..1194: note = Artificial Sequence
  source 1..1194: mol_type = other DNA